(19) United States
(12) Patent Application Publication
(10) Pub. No.: US 2011/0229423 A1
(43) Pub. Date: Sep. 22, 2011

(54) PACAP SIGNALING PATHWAY MODULATORS FOR TREATING INFLAMMATORY SKIN DISEASES HAVING A NEUROGENIC COMPONENT, NOTABLY ROSACEA, AND COMPOSITIONS COMPRISED THEREOF

(75) Inventors: Johannes Voegel, Grasse (FR); Michel Rivier, Nice (FR); Jerome Aubert, Grasse (FR); Martin Steinhoff, San Francisco, CA (US)

(73) Assignee: Galderma Research & Development, Biot (FR)

(21) Appl. No.: 13/054,769

(22) PCT Filed: Jul. 17, 2009
(86) PCT No.: PCT/EP09/59265

§ 371 Date: May 9, 2011

Related U.S. Application Data
(60) Provisional application No. 61/082,037, filed on Jul. 18, 2008.

(51) Int. Cl. A61K 31/713; G01N 33/566; C12Q 1/68; C07H 21/02; A61P 29/00; A61P 17/10; A61P 17/02; A61P 17/00; A61P 31/00; A61P 31/04; A61P 31/12; A61P 33/00; A61P 31/10; A61P 23/00; A61P 37/08; A61P 39/06; A61P 37/06; A61P 17/08; A61P 17/12; A61Q 17/04; A61P 3/02

(52) U.S. Cl. 424/59; 435/7.92; 435/6.13; 536/24.5; 436/501; 514/44 A; 435/6.17

(57) ABSTRACT

Modulators of specific PACAP receptors are useful for treating inflammatory skin diseases having a neurogenic component, and more particularly rosacea and/or facial erythema, compositions containing these and screening methods are described for identifying PACAP signaling pathway modulators.

The invention relates to the use of modulators of specific PACAP receptors for treating inflammatory skin diseases with a neurogenic component, and more particularly rosacea and/or facial erythema, composition containing them and screening methods for identifying PACAP signalling pathway modulators.

### BACKGROUND OF THE INVENTION

The present invention relates to the use of modulators of specific PACAP (Pituitary adenylate cyclase activating polypeptide, or ‘PACAP’) receptors for treating inflammatory skin diseases with a neurogenic component, and more particularly acne, rosacea, facial erythema, urticaria and atopic dermatitis.

Another embodiment of this invention relates to topical, ingestible or injectable cosmetic/pharmaceutical/dermatological compositions containing PACAP signalling pathway modulators, for treating inflammatory skin diseases with a neurogenic component, and more particularly acne, rosacea, facial erythema, urticaria and atopic dermatitis.

Another embodiment of this invention relates to the identification and the use of compounds which are modulators of the PACAP signalling pathway proteins, for the treatment of inflammatory skin diseases with a neurogenic component, and more particularly acne, rosacea, facial erythema, urticaria and atopic dermatitis. It also relates to methods for the in vitro diagnosis or in vitro prognosis of these pathologies.

Rosacea is a common, chronic and progressive inflammatory dermatitis associated with vascular instability. It mainly affects the central part of the face and is characterized by redness of the face or hot flushes, facial erythema, papules, pustules and telangiectasia. In serious cases, especially in men, the soft tissue of the nose may swell and produce a bulbous swelling known as rhinophyma.

Rosacea generally occurs between the ages of 25 and 70, and is much more common in people of fair complexion. It more particularly affects women, although this affection is generally more severe in men. Rosacea is chronic and lasts for years with periods of exacerbation and of remission.

Rosacea was originally called ‘acne rosacea’ because its papules (points of slight raising of the skin) and its inflammatory pustules (pus scabs) greatly resemble those of common acne. In contrast with common acne, whose aetiology is based on abnormal keratinization, an increase in sebum production and also bacterial inflammation, the inflammation of rosacea is vascular in nature and is poorly understood. The result of this facial vascular anomaly is a permanent oedema of the dermis, which may be accompanied by an increased colonization with Demodex folliculorum, a mite usually found in the follicles of the face.

The pathogenesis of rosacea is poorly understood. Many factors may be involved without necessarily inducing this complaint. They are, for example, psychological factors, gastrointestinal disorders, environmental factors (exposure to sunlight, temperature, humidity), emotional factors (stress), dietary factors (alcohol, spices), hormonal factors or vascular factors, or even infection with Helicobacter pilori.

Rosacea is characterized by several primary and secondary features, which often occur together. The disease can be grouped in four subtypes, however, patients may have characteristics of more than one rosacea subtype at the same time, and the disease does not necessarily evolve from one subtype to the next (Wilkin et al. Standard classification of rosacea: Report of the National Rosacea Society Expert Committee on the Classification and Staging of Rosacea, 2002, J. Am. Acad. Dermatol. Vol 46, pages 584-587):

### Subtype 1: Erythematotelangiectatic Rosacea

Erythematotelangiectatic rosacea is mainly characterized by flushing and persistent central facial erythema. The appearance of telangiectases is common but not essential for a diagnosis of this subtype. Central facial edema, stinging and burning sensations, and roughness or scaling may also be reported. A history of flushing alone is common among patients presenting with erythematotelangiectatic rosacea.

### Subtype 2: Papulopustular Rosacea

Papulopustular rosacea is characterized by persistent central facial erythema with transient papules or pustules or both in a central facial distribution. However, papules and pustules also may occur periorificially (that is, they may occur in the perioral, perinasal, or periocular areas). The papulopustular subtype resembles acne vulgaris, except that comedones are absent. Rosacea and acne may occur concomitantly, and such patients may have comedones as well as the papules and pustules of rosacea. Burning and stinging sensations may be reported by patients with papulopustular rosacea.

This subtype has often been seen after or in combination with subtype 1, including the presence of telangiectases. The telangiectases may be obscured by persistent erythema, papules, or pustules, and tend to become more visible after successful treatment of these masking components.

### Subtype 3: Phymatous Rosacea

Phymatous rosacea includes thickening skin, irregular surface nodularities, and enlargement. Rhinophyma is the most common presentation, but phymatous rosacea may occur in other locations, including the chin, forehead, cheeks, and ears. Patients with this subtype also may have patulous, expressive follicles in the phymatous area, and telangiectases may be present.

This subtype has frequently been observed after or in combination with subtypes 1 or 2, including persistent erythema, telangiectases, papules, and pustules. In the case of rhinophyma, these additional stigmata may be especially pronounced in the nasal area.

### Subtype 4: Ocular Rosacea

The diagnosis of ocular rosacea should be considered when a patient's eyes have one or more of the following signs and symptoms: watery or bloodshot appearance (interpalpebral conjunctival hyperemia), foreign body sensation, burning or stinging, dryness, itching, light sensitivity, blurred vision, telangiectases of the conjunctiva and lid margin, or lid and periocular erythema. Blepharitis, conjunctivitis, and irregularity of the eyelid margins also may occur. Meibomian gland dysfunction presenting as chalazion or chronic staphylococcal infection as manifested by hordeolum (stye) are common signs of rosacea-related ocular disease. Some patients may have decreased visual acuity caused by corneal complications (punctate keratitis, corneal infiltrates/ulcers, or marginal keratitis). Treatment of cutaneous rosacea alone may be inadequate in terms of lessening the risk of vision loss resulting from ocular rosacea, and an ophthalmologic approach may be needed.

Ocular rosacea is most frequently diagnosed when cutaneous signs and symptoms of rosacea are also present. However, skin signs and symptoms are not prerequisite to the diagnosis, and limited studies suggest that ocular signs and symptoms may occur before cutaneous manifestations in up to 20% of patients with ocular rosacea. Approximately half of these patients experience skin lesions first, and a minority have both manifestations simultaneously.

Concerning for example subtype 1, no efficient treatments are available. Papulopustular rosacea can be treated with topical metronidazole or azelaic acid, as well as systemic antibiotics of the cycline class. Phymatous rosacea of the nose is treated by surgery. In summary, on account of the multi-factor aspect of rosacea, there are a huge number of treatments with relatively weak efficacy for this condition, but the search continues for an effective treatment that is without risk for the patient.

Besides rosacea the implication of the nerve system and neuro-regulatory pathways has been described for several other skin diseases. In particular, sensory fibers have been elucidated to be involved in inflammatory skin diseases such as:
urticaria (Blais, C., Jr., Rouleau, J. L., Brown, N. J., Lepage, Y., Spence, D., Munoz, C., Friborg, J., Geadah, D., Gervais, N., and Adam, A. 1999. Serum metabolism of bradykinin and des-Arg9-bradykinin in patients with angiotensin-converting enzyme inhibitor-associated angioedema. Immunopharmacology 43:293-302; Nussberger, J., Cugno, M., Cicardi, M., and Agostoni, A. 1999. Local bradykinin generation in hereditary angioedema. J Allergy Clin Immunol 104:1321-1322; Doutre, M. 1999. Physiopathology of urticaria. Eur J Dermatol 9:601-605; Singh, L. K., Pang, X., Alexacos, N., Letourneau, R., and Theoharides, T. C. 1999. Acute immobilization stress triggers skin mast cell degranulation via corticotropin releasing hormone, neurotensin, and substance P: A link to neurogenic skin disorders. Brain Behav Immun 13:225-239),
psoriasis (Steinhoff, M., Meinhardt, A., Steinhoff, A., Gemsa, D., Bucala, R., and Bacher, M. 1999. Evidence for a role of macrophage migration inhibitory factor in psoriatic skin disease. Br J Dermatol 141:1061-1066),
atopic dermatitis (Hendrix S, Neuroimmune communication in skin: far from peripheral. J Invest Dermatol. 2008 February; 128(2):260-1; Pincelli, C., Fantini, F., Romualdi, P., Sevignani, C., Lesa, G., Benassi, L., and Giannetti, A. 1992. Substance P is diminished and vasoactive intestinal peptide is augmented in psoriatic lesions and these peptides exert disparate effects on the proliferation of cultured human keratinocytes. J Invest Dermatol 98:421-427; Steinhoff, M., Vergnolle, N., Young, S. H., Tognetto, M., Amadesi, S., Ennes, H. S., Trevisani, M., Hollenberg, M. D., Wallace, J. L., Caughey, G. H., et al. 2000. Agonists of proteinase-activated receptor 2 induce inflammation by a neurogenic mechanism. Nat Med 6:151-158; Steinhoff, M., Neisius, U., Ikoma, A., Fartasch, M., Heyer, G., Skov, P. S., Luger, T. A., and Schmelz, M. 2003. Proteinase-activated receptor-2 mediates itch: a novel pathway for pruritus in human skin. J Neurosci 23:6176-6180).
acne vulgaris: aggravation of the disease due to emotional stress has been described, and the neuropeptide substance P has been demonstrated to induce the production of inflammatory mediators in sebaceous glands (Toyoda M, Nakamura M, Morohashi M. Neuropeptides and sebaceous glands. Eur J. Dermatol. 2002 September-October; 12(5):422-7; Lee W J, Jung H D, Lee H J, Kim B S, Lee S J, Kim D W. Influence of substance-P on cultured sebocytes. Arch Dermatol Res. 2008 Apr. 22.; Toyoda M, Nakamura M, Makino T, Kagoura M, Morohashi M. Sebaceous glands in acne patients express high levels of neutral endopeptidase. Exp Dermatol. 2002 June; 11(3):241-7.
keloid and hypertrophic scar formation: the neurogenic inflammation hypothesis has been discussed in keloid and hypertrophic scar formation (Akaishi S, Ogawa R, Hyakusoku H. Keloid and hypertrophic scar: Neurogenic inflammation hypotheses. Med Hypotheses. 2008; 71(1):32-8).

### SUMMARY OF THE INVENTION

A major object of the present invention is the administration of at least one VPAC1-R or VPAC2-R modulator to a mammalian, notably human patient, for treating the rosacea disease states indicated above.

The present invention features also the formulation of at least one VPAC1-R or VPAC2-R modulator into a cosmetic, pharmaceutical or dermatological composition, for treating skin redness of neurogenic origin, in particular due to the release of neuropeptides.

A further object of the present invention is the administration of one or more VPAC1-R or VPAC2-R modulators to a human patient, for treating acne vulgaris, atopic dermatitis, urticaria, keloids or hypertrophic scars.

The invention relates to an in vitro method of screening for compounds which are candidates for the preventive or curative treatment of the above mentioned diseases, which comprises determining the ability of a compound to modulate the expression or the activity of the PACAP signalling pathway proteins. Furthermore, the invention relates to the use of modulators of the expression or of the activity of these proteins for the treatment of the above mentioned diseases. Finally, the invention also relates to methods for the in vitro diagnosis or prognosis of these pathologies.

### DETAILED DESCRIPTION OF BEST MODE AND SPECIFIC/PREFERRED EMBODIMENTS OF THE INVENTION

The neuropeptide PACAP affects the human cutaneous vascular system and causes a marked vasodilatation and flush phenomenon as well as hyperthermia. Therefore, skin sensory nerves contribute to vascular regulation in humans and PACAP may be an essential mediator of neurovascular interactions during health and disease. Thus, modulation and/or inhibition of this potent neuropeptide and its receptors may be a novel target for the treatment of inflammatory skin diseases with a neurogenic component like those mentioned above.

Pituitary adenylate cyclase activating polypeptide (PACAP) is a regulatory peptide which belongs to the VIP/secretin family. Two forms can be distinguished, PACAP-38 and a truncated product PACAP-27, both of which are derived from a 176 precursor protein by posttranslational cleavage (Roosterman et al., 2006, Physiol Rev 85 pages 1309-1379 and references therein). PACAP has 68% identity with vasoactive intestinal peptide (VIP), one of the members of the secretin/glucagon/GHRH family.

PACAP binds to and activates at least three receptors, two of which have the particular feature of also binding VIP. PAC1-R (=PACAP1-R) is a high affinity receptor for PACAP. VPAC1-R (=PACAP2-R=VIP1-R) is a high affinity receptor for VIP and a low affinity receptor for PACAP. VPAC2-R (=PACAP3-R=VIP2-R) is a low affinity receptor for both PACAP and VIP. These receptors are widely distributed in the brain and the peripheral tissues. Therefore a differential and fin-tuned interaction between PACAP and VIP can be suggested.

PACAP displays pleiotropic effects throughout the body during development, but also in adults. It participates in essential functions such as growth, endocrine and digestive activity, cardiovascular and respiratory control, immune responses and circadian rhythm. So far, PACAP has been localized in nerve fibers of various peripheral tissues as well as lymphoid tissues and immunocompetent cells.

Evidence is accumulating that neuropeptides play an essential role in skin-nervous system interactions (Steinhoff, M., Stander, S., Seeliger, S., Ansel, J. C., Schmelz, M., and Luger, T. 2003. Modern aspects of cutaneous neurogenic inflammation. Arch Dermatol 139:1479-1488). Sensory fibers have been elucidated to be involved in inflammatory skin diseases such as urticaria, rosacea or atopic dermatitis (Pincelli, C., Fantini, F., Romualdi, P., Sevignani, C., Lesa, G., Benassi, L., and Giannetti, A. 1992. Substance P is diminished and vasoactive intestinal peptide is augmented in psoriatic lesions and these peptides exert disparate effects on the proliferation of cultured human keratinocytes. J Invest Dermatol 98:421-427).

Moreover, PACAP has been suggested to play an antiinflammatory role during chronic inflammation in experimentally-induced arthritis (Abad, C., Martinez, C., Leceta, J., Gomariz, R. P., and Delgado, M. 2001. Pituitary adenylate cyclase-activating polypeptide inhibits collagen-induced arthritis: an experimental immunomodulatory therapy. J Immunol 167:3182-3189). However, various animal in vivo studies also demonstrated a proinflammatory role of PACAP at early stages of inflammation due to modulation of vasodilatation and plasma extravasation (Roosterman et al., 2006, Physiol Rev 85 pages 1309-1379 and references therein). Thus, PACAP may be involved in vascular and immune responses of human skin.

Also the patent application WO2005/079770 describes the use of a modulator of the high affinity receptor PAC1-R for rosacea treatment.

Surprisingly the Applicant's studies have demonstrated the involvement of the low affinity receptors VPAC1-R or VPAC2-R in vascular regulation in human skin in vivo and indicate that PACAP may be an essential neuromediator of neurovascular interactions during health and diseases like acne vulgaris, psoriasis, atopic dermatitis, urticaria, keloids or hypertrophic scars. The Applicant's studies further demonstrated the involvement of the low affinity receptors VPAC1-R or VPAC2-R in the physiopathology of rosacea, and therefore the usefulness of modulators of VPAC1-R or VPAC2-R in the treatment of this disease.

The invention is directed towards offering a novel method for treating acne vulgaris, atopic dermatitis, urticaria, keloids, hypertrophic scars and particularly rosacea, which consists in administering to an individual suffering from this pathology an effective amount of a modulator of the PACAP signalling pathway.

The invention relates more particularly to the use of a modulator of at least one VPAC1-R or VPAC2-R modulator, for the preparation of a pharmaceutical composition for treating acne vulgaris, atopic dermatitis, urticaria, keloids, hypertrophic scars and particularly rosacea.

According to the present invention, the term ‘receptor modulator’ means any molecule that activates or antagonizes its receptor; the said receptor being VPAC1-R or VPAC2-R.

More particularly, the preferred pharmaceutical composition that is the subject of the present invention is a dermatological composition for topical application to the skin.

According to the present invention, the term ‘treating rosacea’ means the treatment and/or prevention of rosacea, of one or more of the subtypes described above.

According to a first embodiment of the invention, the composition is intended for treating Subtype 1: Erythematotelangiectatic rosacea.

According to a second embodiment of the invention, the composition is intended for treating Subtype 2: Papulopustular rosacea.

According to a third embodiment of the invention, the composition is intended for treating the Subtype 3: Phymatous rosacea.

According to a fourth embodiment of the invention, the composition is intended for treating Subtype 4: Ocular rosacea.

According to further embodiments of the invention, the composition is intended for treating acne vulgaris, atopic dermatitis, urticaria, keloids or hypertrophic scars.

According to a preferential embodiment, the composition contains from 0.0001% to 20% of a modulator as defined above, preferably from 0.001% to 10% and more preferentially from 0.01% to 4% of a modulator as defined above, expressed by weight relative to the total weight of the composition.

Needless to say, the present invention concerns, besides the use of a modulator as defined above, the use of derivatives thereof. The term ‘derivatives’ means compounds that differ from a modulator as defined above by substitution, addition or removal of one or more chemical groups.

The compositions of the invention may also comprise any additive usually used in the pharmaceutical or dermatological field that is compatible with a modulator as defined above. Mention may be made especially of sequestrants, antioxidants, sunscreens, preserving agents, for example DL-alpha-tocopherol, fillers, electrolytes, humectants, dyes, common mineral or organic acids or bases, fragrances, essential oils, cosmetic active agents, moisturizers, vitamins, essential fatty acids, sphingolipids, self-tanning compounds such as DHA, skin calmative and protective agents such as allantoin, pro-penetrating agents and gelling agents. Needless to say, a person skilled in the art will take care to select this or these optional additional compound(s), and/or the amount thereof, such that the advantageous properties of the composition according to the invention are not, or are not substantially, adversely affected.

These additives may be present in the composition in a proportion of from 0 to 20% by weight relative to the total weight of the composition.

Examples of sequestrants that may be mentioned include ethylenediaminetetraacetic acid (EDTA), and also derivatives or salts thereof, dihydroxyethylglycine, citric acid and tartaric acid, or mixtures thereof.

Examples of preserving agents that may be mentioned include benzalkonium chloride, phenoxyethanol, benzyl alcohol, diazolidinylurea and parabens, or mixtures thereof.

Examples of humectants that may be mentioned include glycerol and sorbitol.

The compositions of the invention may contain one or more pro-penetrating agents in preferential concentrations ranging from 0 to 20% and more preferentially ranging from 0.6% to 3% by weight relative to the total weight of the composition. Among the pro-penetrating agents that are preferentially used, without this list being limiting, are compounds such as propylene glycol, dipropylene glycol, propylene glycol dipelargonate, lauroglycol and ethoxydiglycol.

Advantageously, the compositions according to the invention may also contain one or more wetting liquid surfactants in preferential concentrations ranging from 0 to 10% and more preferentially ranging from 0.1% to 2%.

The compositions of the present invention may be in any galenical form normally used for topical application, especially in the form of aqueous, aqueous-alcoholic or oily solutions, dispersions of the lotion type, aqueous, anhydrous or lipophilic gels, emulsions of liquid or semi-liquid consistency of the milk type, obtained by dispersing a fatty phase in an aqueous phase (O/W) or, conversely, (W/O), or suspensions or emulsions of soft, semi-solid or solid consistency of the cream, gel or ointment type, or alternatively microemulsions, microcapsules, microparticles or vesicular dispersions of ionic and/or nonionic type.

Preferably, the creams may be formulated from a mixture of mineral oil or from a mixture of beeswax and of water, which emulsifies instantaneously, to which is added the modulator as defined above, dissolved in a small amount of oil such as almond oil.

The ointments may be formulated by mixing a solution of the said modulator in an oil such as almond oil in warmed paraffin, followed by leaving the mixture to cool.

As examples of compositions according to the invention, mention may be made of those comprising an active phase containing (expressed as weight percentages): 0 to 90%, preferentially 5% to 25% and especially 10% to 20% of water; 0 to 10%, preferentially 0 to 2% and especially 0 to 0.5% of wetting liquid surfactant: 0 to 20%, preferentially 0 to 10% and especially 2% to 5% of pro-penetrating agent; 0.0001% to 20% of a modulator as defined above, preferably from 0.001% to 10% and more preferentially from 0.01% to 1% of a modulator as defined above; and an aqueous phase comprising a pH-independent gelling agent, and water.

The aqueous phase of a composition according to the invention in the form of an emulsion may comprise water, a floral water such as cornflower water or a natural spring or mineral water chosen, for example, from eau de Vittel, the waters of the Vichy basin, eau d'Uriage, eau de la Roche Posay, eau de la Bourboule, eau d'Enghien-les-Bains, eau de Saint Gervais-les-Bains, eau de Néris-les-Bains, eau d'Allevard-les-Bains, eau de Digne, eau de Maizières, eau de Neyrac-les-Bains, eau de Lons-le-Saunier, les Eaux Bonnes, eau de Rochefort, eau de Saint Christau, eau des Fumades, eau de Tercis-les-Bains, eau d'Avène and eau d'Aix-les-Bains.

The said aqueous phase may be present in a content of between 10% and 90% by weight and preferably between 20% and 80% by weight relative to the total weight of the composition.

Non-limiting examples that may be mentioned include gelling agents of the polyacrylamide family such as the sodium acryloyldimethyltaurate copolymer/isohexadecane/polysorbate-80 mixture sold under the name Simulgel 600 by the company SEPPIC, the polyacrylamide/C13-14 isoparaffin/laureth-7 mixture, for instance the product sold under the name Sepigel 305 by the company SEPPIC, the family of acrylic polymers coupled to hydrophobic chains, such as the PEG-150/decyl/SMDI copolymer sold under the name Aculyn 44 (polycondensate comprising at least, as components, a polyethylene glycol containing 150 or 180 mol of ethylene oxide, decyl alcohol and methylenebis(4-cyclohexyl isocyanate) (SMDI), at 35% by weight in a mixture of propylene glycol (39%) and water (26%)), and the family of modified starches such as the modified potato starch sold under the name Structure Solanace, or mixtures thereof.

The preferred gelling agents are derived from the polyacrylamide family, such as Simulgel 600 or Sepigel 305 or mixtures thereof.

The gelling agent as described above may be used in preferential concentrations ranging from 0.1% to 15% and more preferentially ranging from 0.5% to 5%.

The gels may preferably be prepared by dispersing or dissolving the modulator as defined above in a suitable ratio in a gel of carbomer, poloxamer or cellulose-based type.

### Diagnostic Applications

A subject of the invention relates to an in vitro method for diagnosing or monitoring the progression of rosacea and/or facial erythema in an individual, which comprises comparing the expression or activity of at least one of the PACAP signalling pathway proteins, the expression of its gene or the activity of at least one of its promoters, in a biological sample from an individual, relative to a biological sample from a control individual.

The expression of the proteins can be determined by assaying the PACAP signalling pathway proteins according to one of the methods such as Western blotting, immunohistochemistry, analysis by mass spectrometry (Maldi-TOF and LC/MS analysis), radioimmunoassay (RIA) and ELISA or any other method known to those skilled in the art. Another method, in particular for measuring the expression of a PACAP signalling pathway gene, is to measure the amount of the corresponding mRNA by any method such as RT-PCR, ribonuclease protection assay, northern blotting, hybridisation-based microarray technologies or any other method known to those skilled in the art. Assaying the activity of PACAP signalling pathway can also be envisaged.

In the context of a diagnosis, the “control” individual is a “healthy” individual. In the context of monitoring the progression of rosacea and/or facial erythema, the “control individual” refers to the same individual at a different time, which preferably corresponds to the beginning of the treatment (TO). This measurement of the difference in expression or in activity of at least one of the PACAP signalling pathway proteins, or in expression of its gene or in activity of at least one of its promoters, makes it possible in particular to monitor the efficacy of a treatment, in particular a treatment with a PACAP signalling pathway modulator, as envisaged above, or another treatment against rosacea and/or facial erythema. Such monitoring can reassure the patient as to the well-founded grounds or the need for continuing this treatment.

Another aspect of the present invention relates to an in vitro method for determining an individual's susceptibility to developing rosacea and/or facial erythema, which comprises comparing the expression or the activity of at least one of the PACAP signalling pathway proteins, the expression of its gene or the activity of at least one of its promoters, in a biological sample from an individual, relative to a biological sample from a control individual.

Here again, the expression of the PACAP signalling pathway proteins can be determined by assaying this protein by immunoassay, for example by ELISA assay or by any other method mentioned above. Another method, in particular for measuring the expression of the PACAP signalling pathway gene, is to measure the amount of corresponding mRNA by any method as described above. Assaying the activity of PACAP signalling pathway can also be envisaged.

The individual tested here is an asymptomatic individual who does not show any skin condition linked to rosacea and/or facial erythema. The “control” individual in this method means a “healthy” reference individual or population. The detection of this susceptibility makes it possible to set up a preventive treatment and/or increased monitoring of the signs associated with rosacea and/or facial erythema.

In these in vitro diagnostic or prognostic methods, the biological sample tested may be any sample of biological fluid or a tissue sample obtained by invasive or non-invasive methods. Without being exhaustive, and without excluding other approaches known to the man of the art, these include for example biopsies (invasive method), tape stripping or hair/beard follicle plucking (non-invasive methods).

### Screening Methods

A subject of the invention is an in vitro or in vivo method of screening for compounds which are candidates for the preventive and/or curative treatment of rosacea and/or facial erythema, which comprises determining the ability of a compound to modulate the expression or the activity of at least one of the PACAP signalling pathway proteins or the expression of its gene or the activity of at least one of its promoters, said modulation indicating the usefulness of the compound for the preventive or curative treatment of rosacea and/or facial erythema. The method therefore makes it possible to select the compounds capable of modulating the expression or the activity of at least one of the PACAP signalling pathway proteins, or the expression of its gene, or the activity of at least one of its promoters.

More particularly, the subject of the invention is an in vitro method of screening for compounds which are candidates for the preventive and/or curative treatment of rosacea and/or facial erythema, which comprises the following steps:
a. preparing at least two biological samples or reaction mixtures;
b. bringing one of the samples or reaction mixtures into contact with one or more of the test compounds;
c. measuring the expression or the activity of at least one of the PACAP signalling pathway proteins, the expression of its gene or the activity of at least one of its promoters, in the biological samples or reaction mixtures;
d. selecting the compounds for which a modulation of the expression or of the activity of at least one of the PACAP signalling pathway proteins, of the expression of its gene or of the activity of at least one of its promoters, is measured in the sample or the mixture treated in b), relative to the non-treated sample or mixture.

An in vivo screening method can be carried out in any laboratory animal, for example a rodent. According to a preferred embodiment, the screening method comprises administering the test compound to the animal, preferably by topical application, then optionally sacrificing the animal by euthanasia and taking an epidermal sheet sample before evaluating the expression of the gene in the epidermal sheet, by any method described herein.

The term “modulation” is intended to mean any effect on the expression or the activity of the protein, the expression of the gene or the activity of at least one of its promoters, i.e. optionally a partial or complete stimulation, but preferably a partial or complete inhibition. Thus, the compounds tested in step d) above preferably inhibit the expression or the activity of at least one of the PACAP signalling pathway proteins, the expression of its gene or the activity of at least one of its promoters. The difference in expression obtained with the compound tested, compared with the control carried out in the absence of the compound, is significant starting from 25% or more.

Throughout the present text, unless otherwise specified, the term “expression of a gene” is intended to mean the amount of mRNA expressed.

The term “expression of a protein” is intended to mean the amount of this protein.

Without being exhaustive, and without excluding other effects known to the man of the art, the term “activity of the PACAP signalling pathway proteins” is intended to mean the ability of the proteins to induce vascular effects, including erythema and edema, to modulate nitric oxide production, or to induce cAMP synthesis.

The term “activity of a promoter” is intended to mean the ability of this promoter to trigger the transcription of the DNA sequence encoded downstream of this promoter (and therefore indirectly the synthesis of the corresponding protein).

The compounds tested may be of any type. They may be of natural origin or may have been produced by chemical synthesis. They may be a library of structurally defined chemical compounds, of non-characterized compounds or substances, or a mixture of compounds.

Various techniques can be used to test these compounds and to identify the compounds of therapeutic interest which are modulators of the expression or of the activity of the PACAP signalling pathway proteins.

According to a first embodiment, the biological samples are cells transfected with a reporter gene functionally linked to all or part of the promoter of the gene encoding the PACAP signalling pathway proteins, and step c) described above consists in measuring the expression of said reporter gene.

The reporter gene may in particular encode an enzyme which, in the presence of a given substrate, leads to the formation of coloured products, such as CAT (chloramphenicol acetyltransferase), GAL (beta-galactosidase) or GUS (beta-glucuronidase). It may also be the luciferase gene or the GFP (Green Fluorescent Protein) gene. The protein encoded by the reporter gene, or the activity thereof, is assayed conventionally by colorimetric, fluorometric or chemiluminescence techniques, inter alia.

According to a second embodiment, the biological samples are cells expressing the gene encoding the PACAP signalling pathway proteins, and step c) described above consists in measuring the expression of said gene.

The cell used here may be of any type. It may be a cell endogenously expressing the PACAP signalling pathway gene, for instance an endothelial cell, or better still a human microvascular endothelial cell. Organs of human or animal origin may also be used, for instance skin preparations.

It may also be a cell transformed with a heterologous nucleic acid, encoding the PACAP signalling pathway proteins, preferably human or mammalian PACAP signalling pathway proteins.

A large variety of host-cell systems can be used, such as, for example, Cos-7, CHO, BHK, 3T3 or HEK293 cells. The nucleic acid may be transfected stably or transiently, by any method known to those skilled in the art, for example by calcium phosphate, DEAE-dextran, liposome, virus, electroporation or microinjection.

In these methods, the expression of the PACAP signalling pathway gene or of the reporter gene can be determined by evaluating the amount of transcription of said gene, or the amount of translation thereof.

The expression “amount of transcription of a gene” is intended to mean the amount of corresponding mRNA produced. The expression “amount of translation of a gene” is intended to mean the amount of protein produced.

Those skilled in the art are familiar with techniques for the quantitative or semi-quantitative detection of the mRNA of a gene of interest. Techniques based on hybridation of the mRNA with specific nucleotide probes are the most common (Northern blotting, RT-PCR (Reverse Transcriptase Polymerase Chain Reaction), quantitative RT-PCR (qRT-PCR), RNase protection). It may be advantageous to use detectable labels, such as fluorescent, radioactive or enzymatic agents or other ligands (for example, avidin/biotin).

In particular, the expression of the gene can be measured by real-time PCR or by RNase protection. The term “RNase protection” is intended to mean the detection of a known mRNA among the poly(A)-RNAs of a tissue, which can be carried out by means of a specific hybridization with a labelled probe. The probe is a labelled (radioactive) RNA complementary to the messenger to be sought. It may be constructed from a known mRNA, the cDNA of which, after RT-PCR, has been cloned into a phage. Poly(A)-RNA from the tissue in which the sequence is to be sought is incubated with this probe under slow hybridization conditions in a liquid medium. RNA:RNA hybrids form between the mRNA sought and the antisense probe. The hybridized medium is then incubated with a mixture of ribonucleases specific for single-stranded RNA, such that only the hybrids formed with the probe can withstand this digestion. The digestion product is then deproteinated and repurified, before being analysed by electrophoresis. The labelled hybrid RNAs are detected by autoradiography. Alternative non radioactive detection procedures and high throughput type variations of this procedure, termed quantitative nuclease protection assay (qNPA) are known to those skilled in the art.

The amount of translation of the gene is evaluated, for example, by immunoassaying the product of said gene. The antibodies used for this purpose may be of polyclonal or monoclonal type. They are produced by conventional techniques. A polyclonal anti-PACAP signalling pathway antibody can, inter alia, be obtained by immunization of an animal such as a rabbit or a mouse, with the whole protein. The antiserum is sampled and depleted according to methods known per se to those skilled in the art. A monoclonal antibody can, inter alia, be obtained by the conventional method of Köhler and Milstein (Nature (London), 256: 495-497 (1975)). Other methods for preparing monoclonal antibodies are also known. Monoclonal antibodies can, for example, be produced by expression of a nucleic acid cloned from a hybridoma. Antibodies can also be produced by the phage display technique, by introducing antibody cDNAs into vectors, which are typically filamentous phages which present libraries of V genes at the surface of the phage (for example, fUSE5 for E. coli).

The immunoassay may be carried out in the solid phase or in the homogeneous phase; in one step or in two steps; by a sandwich method or by a competitive method, by way of non-limiting examples. According to a preferred embodiment, the capture antibody is immobilized on a solid phase. By way of non-limiting examples of a solid phase, use may be made of microplates, in particular polystyrene microplates, or solid particles or beads, or paramagnetic beads.

ELISA assays, radioimmunoassays, or any other detection technique can be implemented in order to reveal the presence of the antigen-antibody complexes formed.

The characterization of the antigen/antibody complexes, and more generally of the isolated or purified, but also recombinant, proteins (obtained in vitro and in vivo) can be carried out by mass spectrometry analysis. This identification is made possible through the analysis (determination of the mass) of the peptides generated by enzymatic hydrolysis of the proteins (in general, trypsin). The proteins are, in general, isolated according to methods known to those skilled in the art, prior to the enzymatic digestion. The analysis of the peptides (in hydrolysate form) is carried out by separation of the peptides by HPLC (nano-HPLC) based on their physicochemical properties (reverse phase). The determination of the mass of the peptides thus separated is carried out by ionization of the peptides and either by direct coupling to the mass spectrometer (ESI electrospray mode), or after deposition and crystallization in the presence of a matrix known to those skilled in the art (analysis in MALDI mode). The proteins are then identified using appropriate software (for example Mascot).

According to a third embodiment, the screening method comprises bringing a compound into contact with a PACAP signalling pathway protein and determining the ability of the compound to modulate the activity of PACAP signalling pathway, a difference in activity, compared with a control carried out in the absence of the compound, indicating the usefulness of the compound for the preventive or curative treatment. of inflammatory skin diseases with a neurogenic component, and more particularly acne, rosacea, facial erythema, urticaria and atopic dermatitis.

Preferably, the ability of the compound to bind to the PACAP signalling pathway proteins is also evaluated.

The determination of the activity of PACAP signalling pathway can be carried out in various ways, in particular by revealing the PACAP signalling pathway-induced cAMP production. For example, Human Dermal Microvascular Endothelial cells (HDMEC) can be grown in endothelial cell growth medium. Lyophilized PACAP₁₋₃₈ can be diluted in the appropriate volume of HDMEC assay medium immediately prior to use. IBMX (3-Isobutyl-1-Methylxanthin) can be added to the medium as a phosphodiesterase inhibitor in order to measure all the intracellular built cAMP after receptor stimulation without the intrinsic phosphdiesterase cleaving some of it. After 24 h cells can be harvested and extracted with 65% ethanol in PBS which can be lyophilized and the cAMP concentration can be examined using a cAMP EIA-direct assay (Amersham Pharmacia Biotech).

In an alternative approach, the determination of the activity of PACAP signalling pathway can be carried out using an Enzyme-Linked Immunosorbent Assay (ELISA) for nitric oxide (NO) production in HDMEC. To study cytokine release by HDMEC after PACAP stimulation, cells can be stimulated as described above and supernatants can be collected and frozen until use for ELISA assays. For the detection of NO release, NO-ELISA kits, for example from BioSource Int. can be used.

The compounds selected by the screening methods defined here can then be tested on other in vitro models and/or in vivo models (in animals or humans), for their effects on acne, rosacea, facial erythema, urticaria and atopic dermatitis

### Modulators of the Protein

A subject of the invention is also the use of a modulator of the human PACAP signalling pathway proteins, which can be obtained by means of one of the methods above, for the preparation of a medicament for use in the preventive and/or curative treatment of rosacea and/or facial erythema.

A method of preventive and/or curative treatment of rosacea and/or facial erythema is thus described here, which method comprises the administration of a therapeutically effective amount of a PACAP signalling pathway-protein modulator to a patient requiring such a treatment.

Preferably, the modulator is a PACAP signalling pathway inhibitor. The term “inhibitor” refers to a chemical compound or substance which eliminates or substantially reduces the biological activity of the PACAP signalling pathway proteins. The term “substantially” signifies a reduction of at least 25%, preferably of at least 35%, even more preferably of at least 50%, and more preferably at least 70% or 90%.

A preferred inhibitor interacts with PACAP signalling pathway in solution at inhibitor concentrations of less than 20 μM, less than 10 μM, less than 5 μM, less than 1 μM, preferably less than 0.1 μM, more preferably less than 0.01 μM.

The modulator compound may be an inhibitory anti-PACAP signalling pathway antibody, preferably a monoclonal antibody. Advantageously, such an inhibitory antibody is administered in sufficient amount to obtain a plasma concentration of approximately 0.01 μg per ml to approximately 100 μg/ml, preferably of approximately 1 μg per ml to approximately 5 μg/ml.

The modulator compound may also be a polypeptide, an antisense DNA or RNA polynucleotide, an si-RNA, or a PNA (Peptide nucleic acid, polypeptide chain substituted with purine and pyrimidine bases, the spatial structure of which mimics that of the DNA and allows hybridization thereto).

The modulator compound may also be an aptamer. The aptamer is a class of molecules representing, in terms of molecular recognition, an alternative to antibodies. They are oligonucleotide sequences having the ability to recognize virtually all classes of target molecules with high affinity and specificity. Such ligands can be isolated by systematic evolution of ligands by exponential enrichment (SELEX) of a random sequence library as described by Tuerk and Gold, 1990. The random sequence library can be obtained by combinatorial chemical DNA synthesis. In this library, each member is an optionally chemically modified linear oligomer of a single sequence. Possible modifications, uses and advantages of this class of molecules have been reviewed in Jayasena, 1999, Clinical Chemistry 45(9):1628-1650.

Other advantages and characteristics of the invention will emerge from the examples below:

### Peripheral Effects of PACAP in Human Skin In Vivo—Skin Temperature and Vasodilatation.

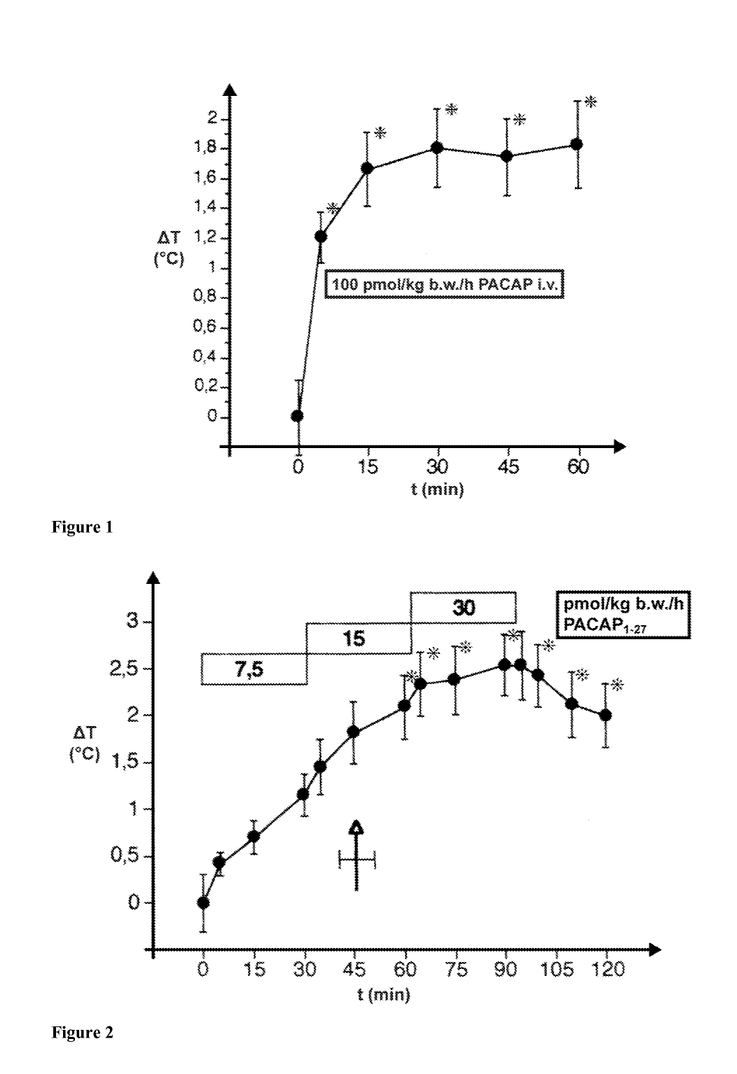

First, the skin temperature (ST) was measured in different human body regions before and after continuous intravenous application of 100 pmol/kg b.w./h PACAP₁₋₂₇. Under basal conditions a ST of 33.51±0.25° C. was measured at 0 min. Differences of ST [ΔT(° C.)] were measured after 5, 15, 30, 45, 60 min (FIG. 1: Changes in skin temperature [ΔT(° C.)] as compared to basal (0=33.51±0.25° C.) before and after continuous infusion of 100 pmol/kg b.w./h PACAP₁₋₂₇ Mean±SEM, *=p<0.05 (n=10)). Our results clearly show that PACAP induced a significant increase of ST already after 5 min (+1.2° C.±0.15). This increase persisted even after 30 min although less dramatic (+1.8° C.±0.26). Between 30 to 60 min after infusion, no significant increase of ST was observed as compared to 30 min.

This PACAP-induced effect in human skin was dose-dependent (FIG. 2: Changes in skin temperature [ΔT(° C.)] as compared to basal after continuous infusion of 7.5, 15, or 30 pmol/kg b.w./h PACAP₁₋₂₇. Mean±SEM, *=p<0.05 (n=9). Mean time until first flush symptoms occurred was 46.03±5.30 min.). I.v. infusion of 7.5 pmol/kg b.w./h PACAP₁₋₂₇ over 30 min resulted in a significant increase of ST (+1.25° C.±0.15) as compared to basal (32.8° C.±0.25). 15 pmol/kg b.w./h PACAP₁₋₂₇ increased ST up to (+2.1° C.±0.2) while 30 pmol/kg b.w./h PACAP₁₋₂₇ effected ST with an increase of +2.4° C.±0.3). Of note, immediately after the infusion of 30 pmol/kg b.w./h PACAP₁₋₂₇ was stopped, the ST slowly decreased.

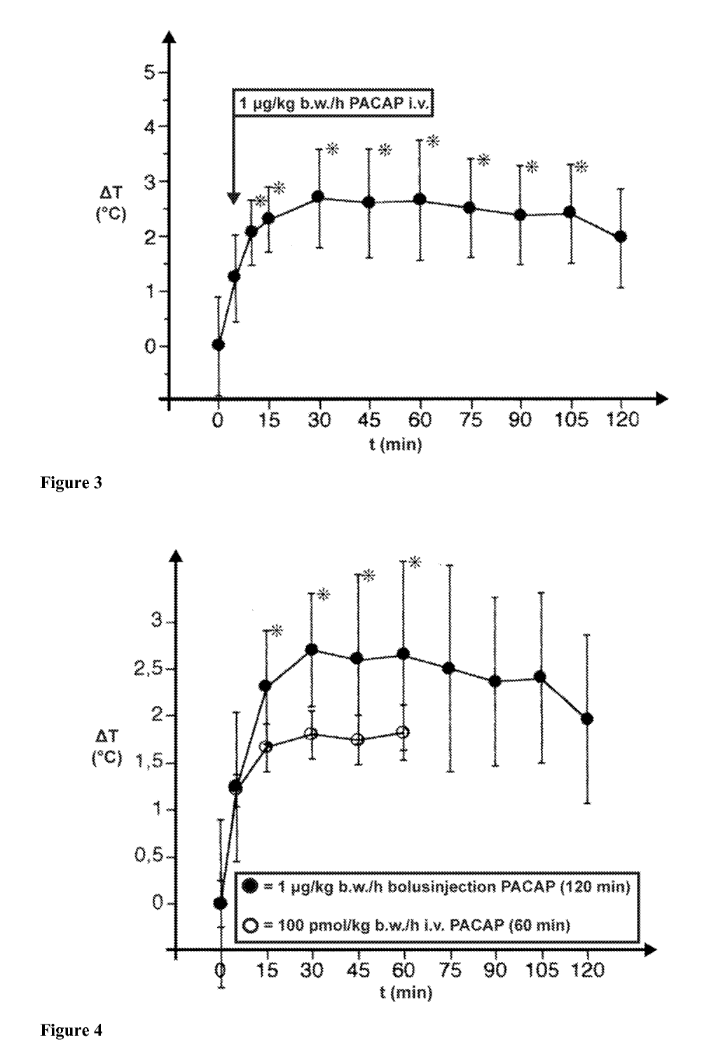

Next, ST was measured in different body regions before and after a bolus injection of 1 μmol/kg b.w./h PACAP₁₋₂₇. Bolus injection also resulted in a rapid and significant increase of ST with a maximum after 30 min (+2.7° C.±0.9) with a following plateau phase (FIG. 3: Changes in skin temperature [ΔT(° C.)] as compared to basal before and after a bolus injection of 1 μg/kg b.w. h PACAP₁₋₂₇. Mean±SEM, *=p<0.05 (n=8)). After 60 min, a slight decrease of ST was obtained although the ST was still elevated after 120 min of bolus injection as compared to basal.

In comparison to a continuous infusion of 100 pmol/kg b.w./h PACAP₁₋₂₇, a bolus injection of 1 μmol/kg b.w./h PACAP₁₋₂₇ showed a significantly higher increase of ST (FIG. 4: Changes in skin temperature [ΔT(° C.)] as compared to basal (=33.51±0.25° C.) under different conditions of continuous infusion and concentrations (◯=1 μg/kg b.w./h PACAP₁₋₂₇; o=100 pmol/kg b.w./h). Mean±SEM, *=p<0.05 (n=10)).

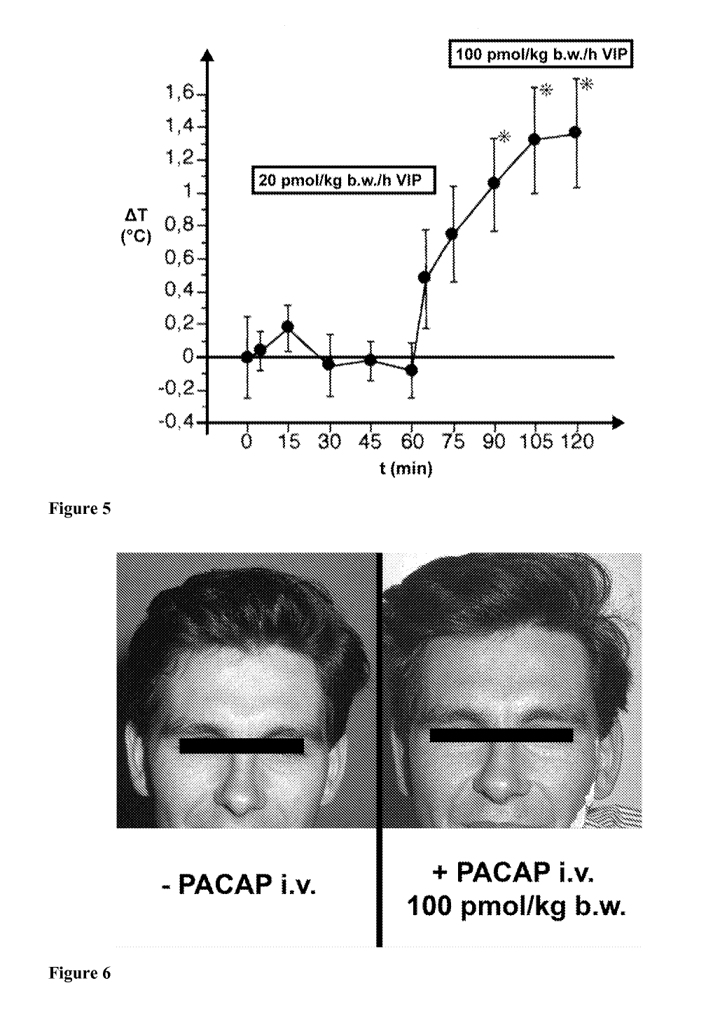

I.v. infusion of 20 pmol/kg b.w./h VIP₁₋₂₈ did not result in a significant increase of ST as compared to basal (32.75° C.±0.25) while infusion of 100 pmol/kg b.w./h VIP₁₋₂₈ resulted in a continuous increase of ST (+1.36° C.±0.33) with a maximum after 60 min, albeit longer time periods were not measured (FIG. 5: Changes in skin temperature [ΔT(° C.)] as compared to basal (32.75±0.25° C.) after continuous infusion of 20 or 100 pmol/kg b.w./h VIP (1-28). Mean±SEM, *=p<0.05 (n=7)).

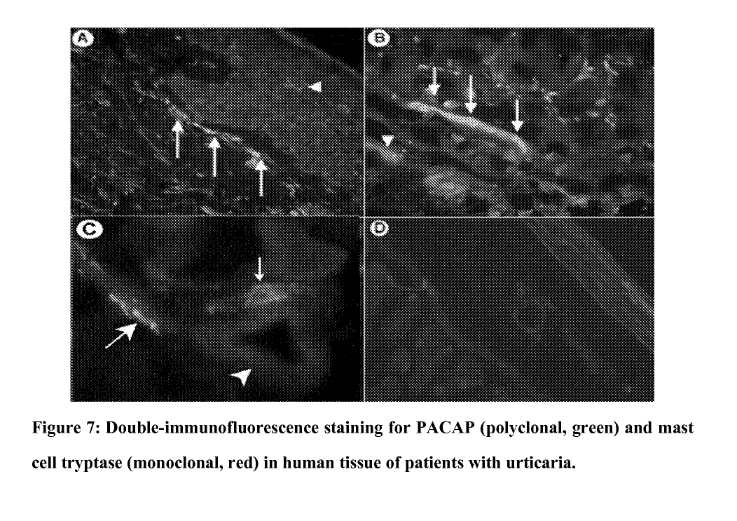

However, in all participants of both experiments (bolus vs. continuous infusion) a significant development of erythema (flushing) and edema was observed as compared to control injections (2% human serum albumin in 0.9% saline) (FIG. 6: A) Normal skin in a probationer before intravenous application of PACAP₁₋₂₇. B) Flush phenomenon in a probationer after intravenous application of PACAP₁₋₂₇. Note cutaneous erythema and facial as well as a marked periorbital edema 30 min after application). This effect was dominant in the face and upper trunk in nine out of ten healthy volunteers. The approximate duration when first signs of a “flush” occurred were 46.03±5.3 min with 100 pmol/kg b.w./h PACAP₁₋₂₇. The erythema persisted approximately for 6 h.

To test whether these prominent effects in human skin (erythema, edema) and difference in skin temperature [ΔT(° C.)] were due to a local vascular effect and not due to a secondary systemic effect we measured systemic parameters such as pulse (B/min) and blood pressure RR syst./diast. (mm Hg). No measurable differences in RR syst./diast. or pulse were observed (not shown). Thus, PACAP-induced changes are most likely due to a local effect in cutaneous vasculature.

Distribution of PACAP in urticaria tissues by double-immunofluorescence. We and others described immunoreactivity for PACAP₁₋₃₈ in normal human skin and its enhanced concentration in patients with psoriasis (Steinhoff, M., McGregor, G. P., Radleff-Schlimme, A., Steinhoff, A., Jarry, H., and Schmidt, W. E. 1999. Identification of pituitary adenylate cyclase activating polypeptide (PACAP) and PACAP type 1 receptor in human skin: expression of PACAP-38 is increased in patients with psoriasis. Regul Pept 80:49-55; Odum, L., Petersen, L. J., Skov, P. S., and Ebskov, L. B. 1998. Pituitary adenylate cyclase activating polypeptide (PACAP) is localized in human dermal neurons and causes histamine release from skin mast cells. Inflamm Res 47:488-492). As demonstrated above, our results clearly show that PACAP is ultimately involved in vasoregulation and erythema of human skin. Consequently, we examined the distribution of PACAP in human skin of patients with urticaria who suffer from acute wheal (edema) and flare (erythema) reactions reflecting vascular reactions such as vasodilatation and plasma extravasation. (FIG. 7: Double-immunofluorescence staining for PACAP (polyclonal, green) and mast cell tryptase (monoclonal, red) in human tissue of patients with urticaria. A) Marked immunostaining for PACAP (green) in nerve fibers close to the dermal-epidermal border of human skin tissue (arrows). In the environment of nerve fibers single tryptase-positive mast cells can be observed (red; ×20). B) In the upper dermis, marked immunofluorescence staining for PACAP in nerve fibers (arrows) closely accompanied by tryptase-positive mast cells (red; ×40). C) PACAP-positive nerve fibers (arrows) close to dermal blood vessels (arrowhead, ×40). D) Negative control (preimmunabsorption) showing absence of PACAP in nerve fibers and endothelial cells demonstrating specificity of immunostaining (×10). Similar to normal human skin, PACAP-positive fibers were found in the dermis of urticaria patients (FIG. 7, arrow) by immunofluorescence.)

Similar to patients with psoriasis, immunopositive staining for PACAP₁₋₃₈ were found in the epidermis (Langerhans cells) of urticaria patients (FIG. 7 A, arrowhead). We also observed a close anatomical association of PACAP-positive nerve fibers (arrows) along with mast cells (FIG. 7 B, arrowheads) indicating that PACAP may induce vasodilatation by activating mast cells. Moreover, PACAP₁₋₃₈ immunoreactivity was observed around dermal blood vessels of urticaria patients (FIG. 7 C). Preabsorption studies prohibited positive staining for PACAP confirming specificity of the immune-reaction (FIG. 7 D).

### Immunohistochemical Detection of VPAC1R in Endothelial Cells of Human Skin Tissues.

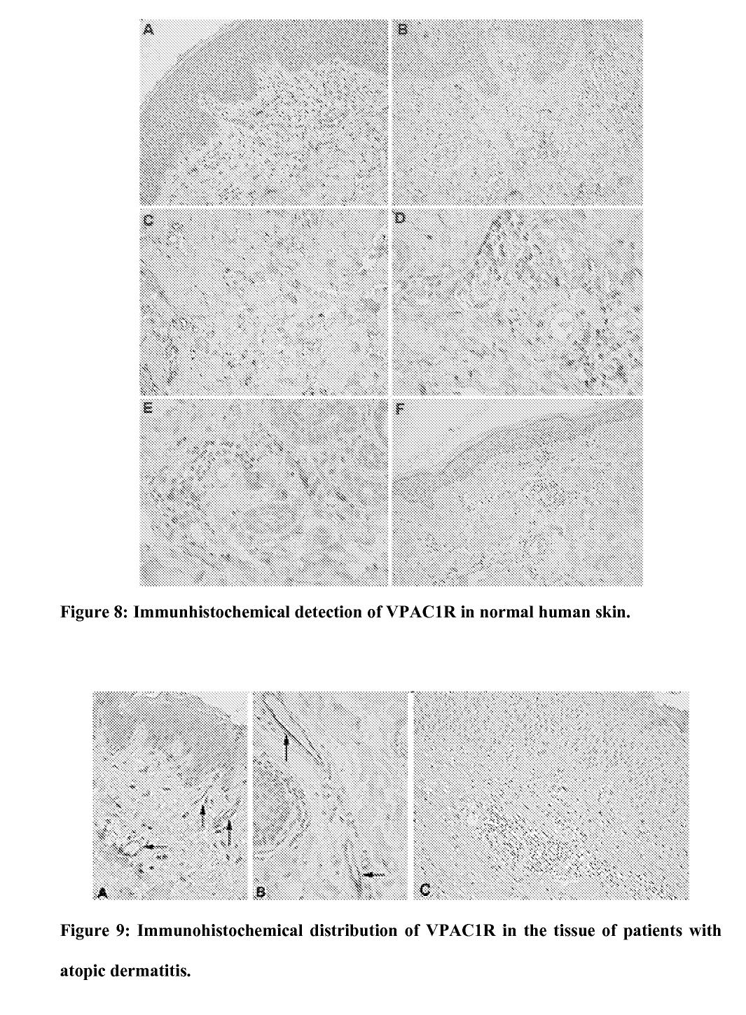

To determine the distribution of VPACRs in normal human skin, we analysed normal skin biopsies by immunohistochemistry. FIG. 8: Immunhistochemical detection of VPAC1R in normal human skin. A) Overview shows dermal staining of VPAC1R in blood vessels and distributed leukocytes. Absence of staining for VPAC1R in keratinocytes as well as fibroblasts (×100). B) Higher magnification shows intensive staining of endothelial cells and monocytes for VPAC1R (×200). C) Weaker staining for VPAC1R in the deeper dermis as compared to superficial dermis (×400). D, E) Negative to weak staining for VPAC1R in dermal connective tissue, sometimes staining of monocytes (×400). F) Control tissue (preimmune absorption) demonstrates absence of VPAC1R in the skin (×100). In summary, VPAC1R in normal human skin demonstrates strongest staining of blood vessels. Higher magnification (FIG. 8 B) reveals intense staining of VPAC1R in dermal endothelial cells and certain leukocytes (macrophages). Background staining for VPAC1R was obtained in keratinocytes.

To determine the distribution of VPACRs in atopic dermatitis, we analysed lesional skin biopsies of atopic dermatitis patients by immunohistochemistry. FIG. 9: Immunohistochemical distribution of VPAC1R in the tissue of patients with atopic dermatitis. A) In atopic dermatitis patients, intensive staining of endothelial cells and leukocytes was observed. Keratinocytes only show a weak staining for VPAC1R in the background range (×100). B) Higher magnification shows intensive staining for VPAC1R in dermal endothelial cells and monocytes (×400). C) Control tissue (preimmune absorption) shows negative staining for VPAC1R (×100). In addition, no immunoreactivity was observed in human skin endothelium using VPAC2R or PAC1R antibodies (data not shown). Thus, VPAC1R is the main receptor for PACAP-induced signalling in human skin endothelial cells and expressed by dermal microvascular endothelial cells during disease state.

### HDMEC Express Functional VPAC1R.

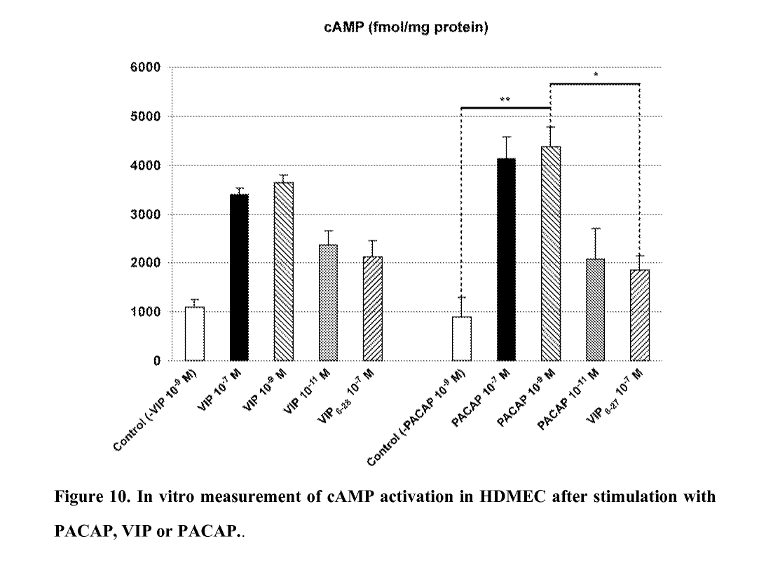

To confirm that HDMEC express functional VPAC1R, we kept HDMEC in FCS/nutrient deficient medium for 24 h and treated them afterwards with specific agonists and/or antagonists (FIG. 10. In vitro measurement of cAMP activation in HDMEC after stimulation with PACAP₁₋₃₈, VIP₆₋₂₈ or PACAP₆₋₂₇. Results show the dose-dependent activation of VPAC1R in HDMEC by PACAP since PAC1R and VPAC2R are not expressed by HDMEC.*: The cAMP activation in HDMEC was cut in half by the application of the specific inhibitor PACAP₆₋₂₇ (=truncated neuropeptide) at a 10-7 M concentration (100× as concentrated as the corresponding agonist in optimal concentration). Pretreatment with antagonist was 3 min before agonist stimulation. This proves the functionality of VPAC1R. **: After the application of PACAP 10⁻⁹ M the cAMP activation in HDMEC was more than 4-fold compared to the negative control. Experiments were performed at least three times). IBMX (3-Isobutyl-1-Methylxanthin; 4 mM) was added to the medium as a phosphodiesterase inhibitor in order to measure all the intracellular built cAMP after receptor stimulation without the intrinsic phosphodiesterase cleaving some of it. The dose-response experiments showed that the concentration of VIP and PACAP were optimal at 10-9 M.

Thus, PACAP induces cAMP activation specifically and effectively in HDMEC. Since HDMEC express VPAC1R but not VPAC2R or PAC1R, VPAC1R may be the receptor which mediates the PACAP-induced responses in human skin.

### PACAP Stimulates HDMEC Cells Via VPAC1R but not VPAC2R and PAC1R

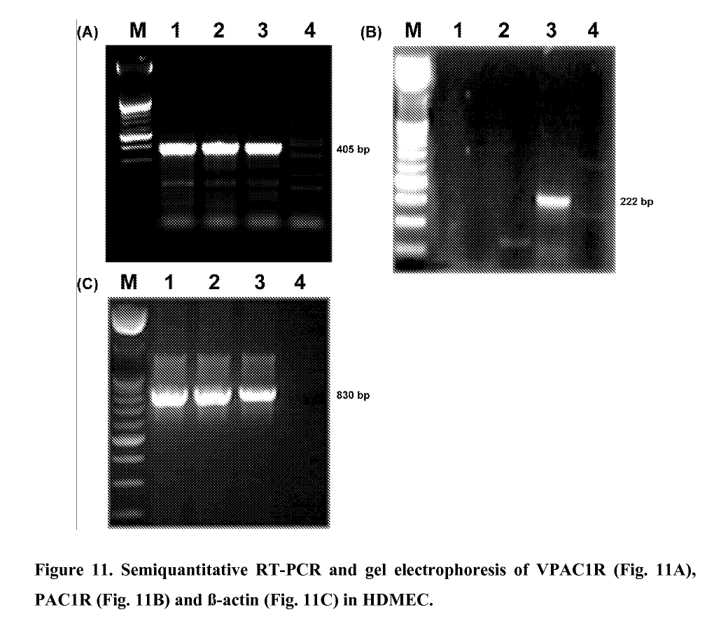

To verify our immunohistochemical observation on the mRNA level that the PACAP-induced effects in human dermal endothelial cells are mediated via VPAC1R, we performed RT PCR analysis for VPAC1R, VPAC2R and PAC1R in HDMEC (FIG. 11. Semiquantitative RT-PCR and gel electrophoresis of VPAC1R (FIG. 11A), PAC1R (FIG. 11B) and β-actin (FIG. 11C) in HDMEC.

M=marker; lane 1: unstimulated HDMEC; lane 2: TNFα stimulated (4 ng/ml) HDMEC; lane 3 (11A): LPS-stimulated (10 ng/ml) HDMEC; lane 3 (11 B and 11C): HNK cells as positive control; lane 4: RT-control (negative control)

In non-stimulated HDMEC cells, mRNA message was only found for PACAP and VPAC1R (FIG. 11A), but not PAC1R (FIG. 11B;) and VPAC2R (data not shown). Note that the PAC1R signal is positive in HNK cells that were used as a positive control. To examine whether proinflammatory mediators such as TNFα or LPS are capable of regulating VPAC1R mRNA expression, HDMEC were harvested without or 6 h after stimulation with TNFα (concentration: 4 ng/ml) or LPS (concentration: 10 ng/ml), respectively, and semiquantitative RT PCR was performed (FIG. 11). To consider the relative mRNA amount in the samples amplification of the housekeeping gene β-actin served as an internal control (FIG. 11C), TNFα as well as LPS induced upregulation of VPAC1R mRNA as compared to the unstimulated controls (FIG. 11). Thus, in contrast to VPAC2R and PAC1R mRNA, VPAC1R mRNA is expressed in HDMEC cells. VPAC1R mRNA can be upregulated in HDMEC cells by proinflammatory mediators such as TNFα or LPS indicating a role of PACAP in inflammatory vascular regulation.

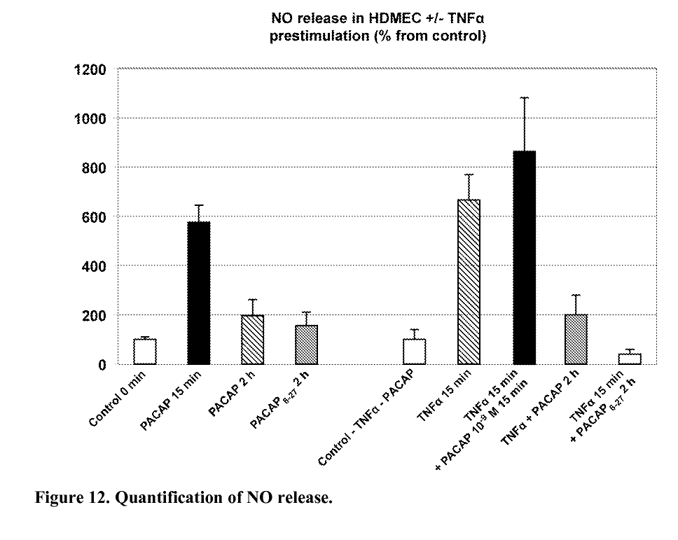

PACAP-induced release of NO in HDMEC. After finding that HDMEC express functional VPAC1R and that VPAC1R is the crucial receptor in human skin epithelium on the protein level, we examined whether PACAP is capable of releasing NO from HDMEC at similar concentrations. Therefore, we performed specific ELISAs with supernatants collected at time-points between 2 h and 12 h (FIG. 12). Indeed, PACAP (10⁻⁹ M) induced the release of NO (582%+/−25) by HDMEC as compared to control with a maximal effect at 15 min. Preincubation using the above described VPAC1R antagonists (PACAP₆₋₂₇) slightly enhanced the PACAP-induced NO release in HDMEC, probably due to its partial agonist effects on VPACRs (79+/−22). When PACAP was incubated with the proinflammatory cytokine TNFα, an additive effect was observed on NO release from HDMEC which was significantly higher than TNFα alone. This effect was significantly lower when TNFa was incubated with the VPAC1R antagonist (PACAP₆₋₂₇) (FIG. 12).

### Expression and Modulation of PACAP and PACAP Receptors in the Skin of Subtype I (Erythematotelangiectatic), Subtype II (Papulopustular) and Subtype III (Phymatous) Rosacea Patients Versus Healthy Volunteers

### Material and Methods:

Skin from healthy patients was obtained following plastic surgery (n=6; face). Biopsies of rosacea were taken from patients with rosacea subtype I (n=6), II (n=6) and III (n=2) (clinical description of each subtype was made according to the classification of Wilkin et al., 2002, J. Am. Acad. Dermatol. Vol 46, pages 584-587) using biopunch, in accordance to good clinical practice. Permission was given by ethical committees in accordance with the ethical standards of the declaration of Helsinki. Skin samples were stored in RNA later and frozen at −80° C. until use. mRNA was prepared using the RNeasy protect Microkit from Qiagen according to the manufacturer's procedure. Quality of mRNA was assessed using the Agilent RNA 6000 NanoKit according to the manufacturer's instructions.

mRNA expression of PACAP, PAC1-R, VPAC1-R and VPAC2-R was evaluated using the semi-quantitative PCR technology (qRT-PCR—Taqman Low Density Arrays). PCR assays were performed in a 7900 HT Cycler (Applied Biosystem). PCR conditions were the following: 40 cycles, 7900 emulation.

Results were expressed in Ct number (Cycle threshold number=number of PCR cycles needed to obtain a significant and quantifiable signal in fluorescence) for each gene. For each group (healthy, rosacea subtype I, II and III), a mean Ct was calculated (arithmetic mean+/−SD). For the interpretation of results, the rule is the following: the mean Ct number is inversely correlated with the mRNA abundance for each gene. A low mean Ct number is the hallmark of a highly expressed gene. Inversely, a high mean Ct number characterizes a very low expressed gene. Following normalization with housekeeping genes (Glyceraldehyde-3-phosphate dehydrogenase (GAPDH), Actin beta (ACTB) and Hypoxanthine phosphoribosyltransferase 1 (HPRT1)), calculation of the relative expression of each gene for the 3 rosacea conditions versus healthy volunteers was performed (mean fold induction—see tables II and III).

Results presented in tables II and III demonstrate the expression of VPAC1-R and VPAC2-R in the skin of all patients at a moderate level (healthy volunteers or rosacea). VPAC1-R and VPAC2-R are weakly modulated, VPAC2-R is moderately induced in patients with rosacea subtype III.

In sum, the neuropeptide PACAP undoubtedly affects the human cutaneous vascular system and causes a marked vasodilatation and flush phenomenon as well as hyperthermia in vivo. Therefore, skin sensory nerves contribute to vascular regulation in humans in vivo and PACAP may be an essential neuromediator of neurovascular interactions during health and diseases like acne vulgaris, atopic dermatitis, urticaria, keloids or hypertrophic scars. Taken together with the results on the expression of PACAP and its receptors in rosacea, these results particularly highlight the importance of PACAP and its receptors in the physiopathology of rosacea and strongly suggest that PACAP and its receptors are important proteins to target in this pathology. In general, inhibition of this potent neuropeptide and its receptors may be a novel target for the treatment of inflammatory skin diseases with a neurogenic component.

### Tables

TABLE I
Measurement of pulse (B/min), systolic (syst) or diastolic
(diast) blood pressure (RR, [mmHg]) at different time
points after infusion of different concentrations of PACAP.

| | pulse | R R systol. | R R diastol. |
|---|---|---|---|
| time (min) | (B/min) | (mmHg) | (mmHg) |
| −15 | 70 ± 4 | 107 ± 3 | 72 ± 3 |
| −10 | 65 ± 3 | 108 ± 4 | 73 ± 4 |
| 0 (7.5 | 67 ± 3 | 108 ± 4 | 73 ± 4 |
| pmol/kg/h) | | | |
| 5 | 64 ± 3 | 107 ± 4 | 72 ± 4 |
| 15 | 66 ± 4 | 109 ± 4 | 75 ± 3 |
| 30 (15 | 68 ± 3 | 111 ± 5 | 75 ± 3 |
| pmol/kg/h) | | | |
| 35 | 69 ± 3 | 111 ± 5 | 74 ± 4 |
| 45 | 73 ± 4 | 111 ± 5 | 74 ± 4 |
| 60 (30 | 69 ± 3 | 111 ± 5 | 74 ± 3 |
| pmol/kg/h) | | | |
| 65 | 71 ± 3 | 109 ± 5 | 74 ± 3 |
| 75 | 72 ± 2 | 110 ± 5 | 74 ± 3 |
| 90 (end of | 69 ± 3 | 109 ± 5 | 72 ± 4 |
| infusion) | | | |
| 100 | 66 ± 4 | 109 ± 5 | 73 ± 4 |
| 110 | 68 ± 4 | 109 ± 4 | 72 ± 4 |
| 120 | 71 ± 4 | 112 ± 4 | 74 ± 3 |

Mean ± SEM,
* = p < 0.05 (n = 10). No measurable differences in RR syst./diast. or pulse were observed. These results indicate that the observed flush effects (erythema, edema) and differences in skin temperature [ΔT(° C.)] were due to a local vascular effect and not due to a secondary systemic effect.

TABLE II
Expression of VPAC1-R and VPAC2-R mRNA in the skin of healthy volunteers and patients with subtype
I (erythematotelangiectatic), subtype II (papulopustular) and subtype III (phymatous) rosacea

| | Healthy Volunteers | | Rosacea Type I | | Rosacea Type II | | Rosacea Type III | |
|---|---|---|---|---|---|---|---|---|
| | | Standard | | Standard | | Standard | | Standard |
| | Mean Ct | deviation | Mean Ct | deviation | Mean Ct | deviation | Mean Ct | deviation |
| GENE NAME | (n = 6) | (Ct) | (n = 6) | (Ct) | (n = 6) | (Ct) | (n = 2) | (Ct) |
| VPAC1-R | 28.41 | 0.63 | 28.88 | 0.62 | 28.72 | 0.59 | 28.39 | 0.42 |
| VPAC2-R | 34.06 | 3.22 | 33.55 | 2.89 | 33.40 | 0.94 | 31.98 | 1.80 |
| ACTB | 22.26 | 0.80 | 21.38 | 1.25 | 21.68 | 1.45 | 21.26 | 1.72 |
| (Housekeeping gene) | | | | | | | | |
| GAPDH | 22.52 | 0.78 | 21.69 | 0.83 | 22.01 | 1.05 | 21.95 | 0.27 |
| (Housekeeping gene) | | | | | | | | |
| HPRT1 | 28.22 | 0.68 | 27.67 | 0.59 | 28.07 | 1.06 | 27.81 | 0.82 |
| (Housekeeping gene) | | | | | | | | |

TABLE III
Modulation VPAC1-R and VPAC2-R mRNA in the skin of patients with
subtype I (erythematotelangiectatic), subtype II (papulopustular)
and subtype III (phymatous) rosacea versus healthy volunteers

MEAN FOLD INDUCTION

| | Healthy | Rosacea | Rosacea | Rosacea |
|---|---|---|---|---|
| Gene Name | Volunteers | Type I | Type II | Type III |
| VPAC1-R | 1.00 | 0.43 | 0.61 | 0.64 |
| VPAC2-R | 1.00 | 0.85 | 1.20 | 2.68 |

1.-38. (canceled)

39. A method for treating an inflammatory skin disease having a neurogenic component, comprises topically administering to a patient in need thereof, a pharmaceutical composition comprising a thus effective amount of a compound that modulates the activity of VPAC1-R or VPAC2-R.

40. The method as defined by claim 39, wherein said inflammatory skin disease is selected from the group consisting of acne, rosacea, facial erythema, urticaria, atopic dermatitis, keloid and hypertrophic scars.

41. The method as defined by claim 39, wherein said pharmaceutical composition comprises a dermatological composition for topical application.

42. The method as defined by claim 39, wherein said composition is for treating at least one subtype of rosacea.

43. The method as defined by claim 39, wherein said composition is for treating the first subtype of rosacea.

44. The method as defined by claim 39, wherein said composition is for treating the second subtype of rosacea.

45. The method as defined by claim 39, wherein said composition is for treating the third subtype of rosacea.

46. The method as defined by claim 39, wherein said composition is for treating the fourth subtype of rosacea.

47. The method as defined by claim 39, wherein said composition comprises from about 0.0001% to 20% of said modulator.

48. The method as defined by claim 39, wherein said composition further comprises another active agent selected from the group consisting of antibiotics, antibacterial agents, antiviral agents, anti-parasitic agents, anti-fungal agents, anesthetics, analgesics, anti-allergic agents, retinoids, free-radical scavengers, anti-pruriginous agents, kerato-lytic agents, anti-seborrhoeic agents, antihistamines, sulfides, immunosuppressant products and anti-proliferative products.

49. The method as defined by claim 39, wherein said composition further comprises an additive selected from the group consisting of sequestrants, antioxidants, sunscreens, preservatives, fillers, electrolytes, humectants, dyes, mineral or organic acids or bases, fragrances, essential oils, cosmetic active agents, moisturizers, vitamins, essential fatty acids, sphingolipids, self-tanning compounds, calmatives and skin-protecting agents, pro-penetrating agents and gelling agents, or mixtures thereof.

50. The method as defined by claim 39, wherein said composition comprises from 0.001% to 10% by weight of said modulator.

51. The method as defined by claim 39, wherein said composition comprises from about 0.01% to 4% by weight of said modulator.

52. An in vitro or in vivo method of screening for compounds which are candidates for the preventive and/or curative treatment of inflammatory skin diseases having a neurogenic component, which comprises determining the ability of a compound to modulate the expression or the activity of the PACAP signaling pathway proteins or the expression of its gene or the activity of at least one of its promoters.

53. The in vitro or in vivo method of screening as defined by claim 52, wherein said inflammatory skin diseases are selected from the group consisting of acne, rosacea, facial erythema, urticaria, atopic dermatitis, keloid and hypertrophic scars, which comprises determining the ability of a compound to modulate the expression or the activity of the PACAP signaling pathway proteins or the expression of its gene or the activity of at least one of its promoters.

54. The in vitro or in vivo method of screening for compounds as defined by claim 52, wherein the PACAP signaling pathway proteins are PACAP, PAC1-R, VPAC1-R and VPAC2-R

55. An in vitro method of screening for compounds which are candidates for the preventive and/or curative treatment of rosacea and/or facial erythema as defined by claim 52, which comprises the following steps:
a. preparing at least two biological samples or reaction mixtures;
b. bringing one of the samples or reaction mixtures into contact with one or more of the test compounds;
c. measuring the expression or the activity of the PACAP signaling pathway proteins, the expression of at least one of its genes or the activity of at least one of its promoters, in the biological samples or reaction mixtures;
d. selecting the compounds for which a modulation of the expression or of the activity of the PACAP signaling pathway proteins, or a modulation of the expression of at least one of its genes or a modulation of the activity of at least one of its promoters, is measured in the sample or the mixture treated in b), relative to the untreated sample or mixture.

56. The method as defined by claim 55, wherein the compounds selected in step d) inhibit the expression or the activity of the PACAP signaling pathway proteins, the expression of its gene or the activity of at least one of its promoters.

57. The method as defined by claim 55, wherein the biological samples are cells transfected with a reporter gene functionally linked to all or part of the promoter of the gene encoding at least one of the PACAP signaling pathway proteins, and step c) comprises measuring the expression of said reporter gene.

58. The method as defined by claim 55, wherein the biological samples are cells expressing the gene encoding at least one of the PACAP signaling pathway proteins, and step c) comprises measuring the expression of said gene.

59. The method as defined by claim 57, in which the cells are cells transformed with a heterologous nucleic acid, encoding at least one of the PACAP signaling pathway proteins.

60. The method as defined by claim 55, wherein the expression of the gene is determined by measuring the amount of transcription of said gene.

61. The method as defined by claim 55, wherein the expression of the gene is determined by measuring the amount of translation of said gene.

62. The method as defined by claim 52, comprises bringing a compound into contact with a at least one of the PACAP signaling pathway proteins and determining the ability of the compound to modulate the PACAP signaling pathway, a difference in activity, compared with a′ control carried out in the absence of the compound, indicating the usefulness of the compound for the preventive or curative treatment of rosacea and/or facial erythema.

63. The method as defined by claim 52, comprising bringing a compound into contact with a at least one of the PACAP signaling pathway proteins and determining the ability of the compound to modulate the PACAP signaling pathway, a difference in activity, compared with a control carried out in the absence of the compound, indicating the usefulness of the compound for the preventive or curative treatment of acne vulgaris, atopic dermatitis, urticaria, keloid or hypertrophic scar formation.

64. A medicament useful for the preventive and/or curative treatment of acne, rosacea, facial erythema, urticaria, atopic dermatitis, keloid and hypertrophic scars, comprising a PACAP signaling pathway modulator obtained by means of the method as defined by claim 52.

65. The medicament as defined by claim 64, in which the modulator is a PACAP signaling pathway-protein inhibitor.

66. The medicament as defined by claim 64, and, in which the modulator is an siRNA.

67. An in vitro method for diagnosing or monitoring the progression of an inflammatory skin disease having a neurogenic component in an individual, which comprises comparing the expression or the activity of the PACAP signaling pathway proteins, the expression of its gene or the activity of at least one of its promoters, in a biological sample from an individual, relative to a biological sample from a control individual.

68. The method as defined by claim 67, wherein said inflammatory skin disease is selected from the group consisting of acne, rosacea, facial erythema, urticaria, atopic dermatitis, keloid and hypertrophic scars

69. The method as defined by claim 67, in which the expression of the protein is determined by assaying this protein by immunoassay.

70. The method as defined by claim 67, in which the immunoassay is an ELISA assay.

71. The method as defined by claim 69, in which the expression of the gene is determined by measuring the amount of corresponding mRNA.

72. An in vitro method for determining an individual's susceptibility to developing an inflammatory skin disease having a neurogenic component, which comprises comparing the expression or the activity of the PACAP signaling pathway proteins, the expression of its gene or the activity of at least one of its promoters, in a biological sample from an individual, relative to a biological sample from a control individual.

73. The method as defined by claim 72, wherein said inflammatory skin disease is selected from the group consisting of acne, rosacea, facial erythema, urticaria, atopic dermatitis, keloid and hypertrophic scars.

74. The method as defined by claim 72, in which the expression of the protein is determined by assaying this protein by immunoassay.

75. The method as defined by claim 72, in which the immunoassay is an ELISA assay or a radioimmunoassay.

76. The method as defined by claim 72, in which the expression of the gene is determined by measuring the amount of corresponding mRNA.
